(19) United States
(12) Patent Application Publication
(10) Pub. No.: US 2007/0239227 A1
(43) Pub. Date: Oct. 11, 2007

(54) Frequency modulated stimulation strategy for cochlear implant system

(76) Inventor: Gene Y. Fridman, Santa Clarita, CA (US)

Correspondence Address: ADVANCED BIONICS CORPORATION, 25129 RYE CANYON ROAD, VALENCIA, CA, 91355, US

(21) Appl. No.: 10/917,789

(22) Filed: Aug. 13, 2004

Related U.S. Application Data
(60) Provisional application No. 60/495,639, filed on Aug. 15, 2003.

(51) Int. Cl. A61N 1/00

(52) U.S. Cl. 607/57

(57) ABSTRACT

A new speech processing strategy, termed Frequency Modulated Stimulation (FMS), is provided for use with a cochlear prosthetic. The FMS strategy advantageously mimics the neural firing patterns of the healthy cochlea by controlling when and where stimulation pulses are presented in the cochlea. The benefits of this approach are its simplicity and its ability to provide temporal information at relatively low power consumption. The stimulation that results has high temporal precision and a low pulse presentation rate. The power efficiency of the FMS strategy is three to six times greater than that of a CIS strategy with comparable thresholds. The FMS strategy depends on the probability that at any point along the basilar membrane the ganglion cells are most likely to respond during the upward motion of the basilar membrane, when the hair cells are pushed toward the tectorial membrane. At low frequencies, this probability accounts for phase locking of the neurons to each peak of the motion. At high frequency locations, phase locking occurs at integer multiples of the vibration cycles because the vibration of the membrane is faster than the refractory period of the neurons. The FMS strategy provided by the invention takes advantage of the natural behavior of the ganglion cells by outputting a biphasic pulse at the preset integer multiples of the vibration cycles. Integer multiples are determined by counting the positive-to-negative zero crossings, or equivalent frequency counting, at the output of the band pass filters that decompose the incoming audio signal(s).

The present invention claims the benefit of U.S. Provisional Patent Application Ser. No. 60/495,639, filed Aug. 15, 2003, which application (including Appendix A of Application Ser. No. 60/495,639) is incorporated herein by reference in its entirety.

### BACKGROUND OF THE INVENTION

The present invention relates to implantable neurostimulation devices and systems, and more particularly to an implantable cochlear stimulation system that aids the profoundly deaf to hear. Even more particularly, the invention relates to a Frequency Modulated Stimulation (FMS) strategy that may be used with such an implantable cochlear stimulation system in order to simplify the signal processing and significantly reduce power consumption.

Currently available stimulation strategies for use with cochlear implants offer either power efficiency, such as the Continuous Interleaved Sampler (CIS) strategy, or a more preferred natural sound, such as the Simultaneous Analog Stimulation (SAS) strategy. Disadvantageously, one strategy is usually used at the expense of the other. The CIS and SAS strategies, and other stimulation strategies, are described in U.S. Pat. Nos. 6,219,580 and 6,289,247, both of which patents are incorporated herein by reference.

What is needed is a new stimulation strategy that better mimics the natural neural firing patterns in a healthy cochlea in order to present stimulation pulses at the proper times and locations in the cochlear implant, and which does so with a significant power efficiency improvement. The FMS strategy of the present invention satisfies this and other needs.

### SUMMARY OF THE INVENTION

The present invention provides a new speech processing strategy, or scheme, termed Frequency Modulated Stimulation (FMS), for use with a cochlear prosthetic. The FMS strategy advantageously mimics the neural firing patterns of the healthy cochlea by controlling when and where stimulation pulses are presented in the cochlea. The benefits of this approach are its simplicity and its ability to provide temporal information at relatively low power consumption. The stimulation that results has high temporal precision and a low pulse presentation rate. The power efficiency of the FMS strategy is three to six times greater than that of a CIS strategy with comparable thresholds.

The spiral ganglion cells at any given location along the basilar membrane are believed to have a maximal firing rate at integer multiples of the vibration period of the basilar membrane at that location. This is referred to as “phase locking.” The maximal firing rate is believed to be proportional to the amplitude of the signal. Furthermore, the time of this maximum response shifts to higher integer multiples with the decrease in signal amplitude. That is, the average time between the responses is longer for quiet signals, and shorter for loud signals.

The FSM strategy of the present invention models the above-described natural average behavior of the ganglion cells by introducing a biphasic stimulation pulse at the times and with the amplitudes that correspond to the maximal average response of the neurons in a healthy cochlea.

More particularly, the FMS strategy of the present invention depends on the probability that at any point along the basilar membrane the ganglion cells are most likely to respond during the upward motion of the basilar membrane, when the hair cells are pushed toward the tectorial membrane. At low frequencies, this probability accounts for phase locking of the neurons to each peak of the basilar membrane motion. At high frequency locations, phase locking occurs at integer multiples of the vibration cycles due to the fact that the vibration of the membrane is faster than the refractory period (RP) of the neurons.

The FMS strategy of the present invention presents a biphasic pulse at preset integer multiples of the vibration cycles. More particularly, the vibration cycles at the higher frequencies (having periods significantly shorter than the refractory period of the neurons) are counted, or monitored, until the total duration of the counted cycles is more or less equal to the refractory period, at which point a stimulus pulse is generated in order to cause the neurons to fire, and to phase lock such firing with the peak of the basilar membrane motion.

In one preferred embodiment, bandpass filters are used to decompose the input sound waves and separate them into respective frequency bands. Each frequency band corresponds to a respective location within the tonotopically-arranged cochlea where one or more stimulation electrodes have been inserted. With such an arrangement, the control algorithm used to implement the FMS strategy of the present invention counts the positive-to-negative zero crossings at the output of the bandpass filters, and when a prescribed count has been reached (corresponding to a time duration, or period, that is about the same as the refractory period of the neurons) generates a stimulation pulse, e.g., a biphasic stimulation pulse, and presents the stimulation pulse to that region of the cochlea that corresponds to the frequency range or band of the bandpass filter.

In accordance with another preferred embodiment, the FMS strategy recognizes that the refractory period (RP) of the neurons is not constant, nor static. Rather, the RP is modulated between about 1 and 5 ms according to the amplitude of the incoming signal. Thus, in a healthy cochlea, a loud signal will, on average, produce a neuronal response every 2 or 3 ms, but a quiet signal of the same frequency will produce a neuronal response only about every 5 ms, but sill always at an integer multiple of the signal period.

Hence, in accordance with this embodiment of the invention, the RP value is treated as a variable and is modulated in accordance with the amplitude of the filtered signal. This causes a stimulation pulse to be generated sooner for a loud signal (i.e., fewer integer multiples of the signal period) than for a quiet signal of the same frequency.

In accordance with another embodiment, the amplitude of the biphasic pulse that is generated in each channel or frequency band corresponds to the highest signal amplitude that has occurred since the previously delivered pulse for that channel. Because the period of the output of each filter varies according to the filter parameters, the duration between pulses also phase locks to these fluctuations as in the healthy cochlea.

In accordance with one aspect of the invention, there is provided a method of stimulating the cochlea using a frequency modulated stimulation (FMS) strategy. Such method includes, as a first step, dividing incoming sound signals into at prescribed number of frequency bands using bandpass filters, e.g., at least four frequency bands and preferably eight or more frequency bands. Each frequency band covers a respective portion of the audio spectrum and also corresponds to an identified region of the tonotopically-arranged cochlea where one or more electrodes are positioned for stimulation.

Each frequency band, i.e., the bandpass filter that is used to define each frequency band, provides an output signal that varies as a function of the sound components present within that frequency band. Thus, a second step of the method of stimulating provided by the invention involves monitoring the number of cycles of the output signal of each frequency band. Such monitoring may be accomplished in various ways, but a preferred approach is to simply count the positive-to-negative zero crossings that occur in the output signal of each frequency band, i.e., that occur in the output signal of each bandpass filter. (Alternatively, the negative-to-positive zero crossings could be counted; or all the zero crossings could be counted, with the result divided by two.)

A third step of the method of stimulating provided by the invention involves generating, for every Nth cycle of the output signal of each frequency band, a biphasic stimulation pulse that is applied through a respective electrode to stimulate an appropriate region of the cochlea, where N represents an integer that roughly defines the number of cycles of the output signal for that frequency band that occur during a predetermined refractory period of the cochlea.

In the manner described above, then, each tonotopic region of the cochlea is stimulated no more than once during the predetermined refractory period as a function of the sound components that occur within the frequency band associated with that region of the cochlea. Advantageously, this strategy not only phase locks the stimuli applied to the cochlea more or less with the basilar membrane vibrations, but it also generates stimulation pulses, for most frequency bands, at a rate that better matches the refractory period of the neurons. This action, in turn, generally results in fewer stimulation pulses being generated than have been used in prior stimulation strategies, e.g., SAS and CIS strategies, thereby reducing power consumption of the cochlear implant system (which reduced power consumption allows the battery or other power source that provides operating power for the implant system to operate for a longer period of time between recharging or replacement). The above-described action also produces stimulation of the neurons in a way that better mimics the operation of a healthy cochlea, thereby producing a more natural sound sensation.

It is thus a feature of the present invention to provide a cochlear implant system that is more efficient in terms of power consumption than prior cochlear implant systems.

It is a further feature of the invention to provide a cochlear implant system that better mimics the operation of a healthy cochlea by stimulating the neurons at a time that is more or less phase locked to the motion of the basilar membrane, and at a rate that is no faster than the refractory period of the neurons.

It is an additional feature of the invention to provide a speech processing strategy that models the average responses of the spiral ganglion cells to pure tones in a healthy cochlea.

It is yet another feature of the invention to provide a speech processing strategy that provides more natural sound at lower power consumption.

### BRIEF DESCRIPTION OF THE DRAWINGS

The above and other aspects, features and advantages of the present invention will be more apparent from the following more particular description thereof, presented in conjunction with the following drawings and appendix wherein:

FIG. 1A shows a generalized block diagram of a cochlear stimulation system, including a microphone, a speech processor (SP), an implantable cochlear stimulator (ICS) and an electrode array;

FIG. 1B is a functional block diagram of one channel of the speech processor and ICS portions of a multichannel cochlear stimulation system;

FIG. 1C depicts a typical filter response of the bandpass filter used in most of the channels of the speech processor of a multichannel cochlear stimulation system;

FIG. 1D presents one exemplary frequency map for the filter bank used within the speech processor for two different speech processing strategies;

FIG. 2 shows a functional block diagram of the invention;

FIG. 3 illustrates one implementation of the FM Processing block shown in FIG. 2;

FIG. 4 shows a schematic representation of a cross sectional view of a human cochlea;

FIG. 5 diagramatically illustrates the normal neural firing patterns that occur within the cochlea;

FIG. 6 illustrates one implementation of the Frequency Modulated Stimulation (FMS) strategy of the present invention; and

FIG. 7 depicts how the FMS strategy of the invention emulates the normal neuron firing pattern of the cochlea.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention in the context of one or more preferred embodiments of the invention. The scope of the invention should be determined with reference to the claims.

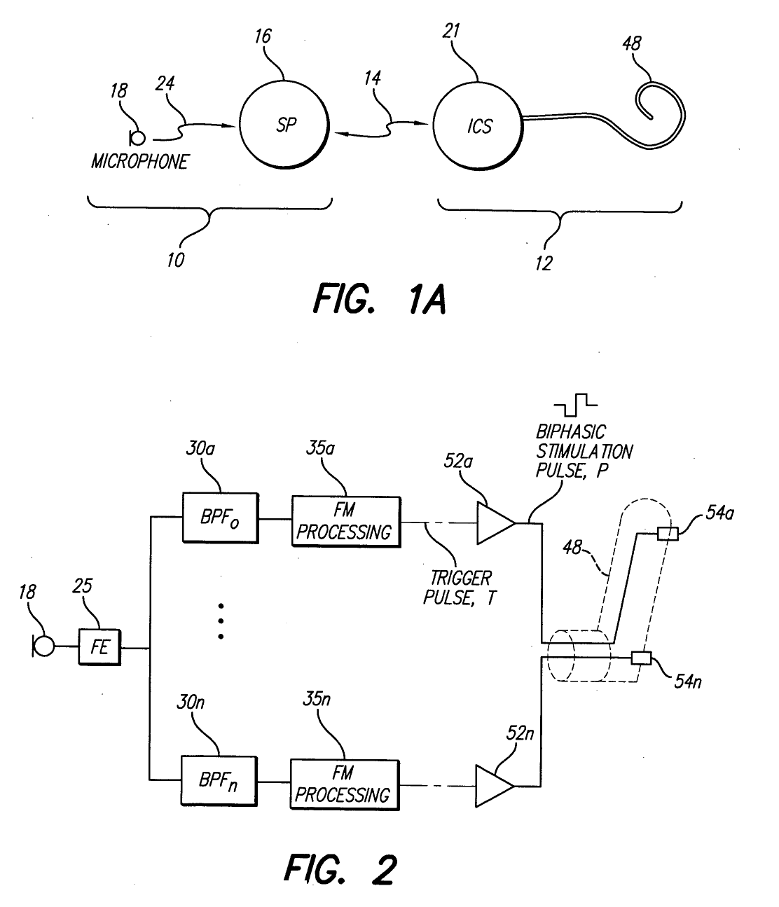

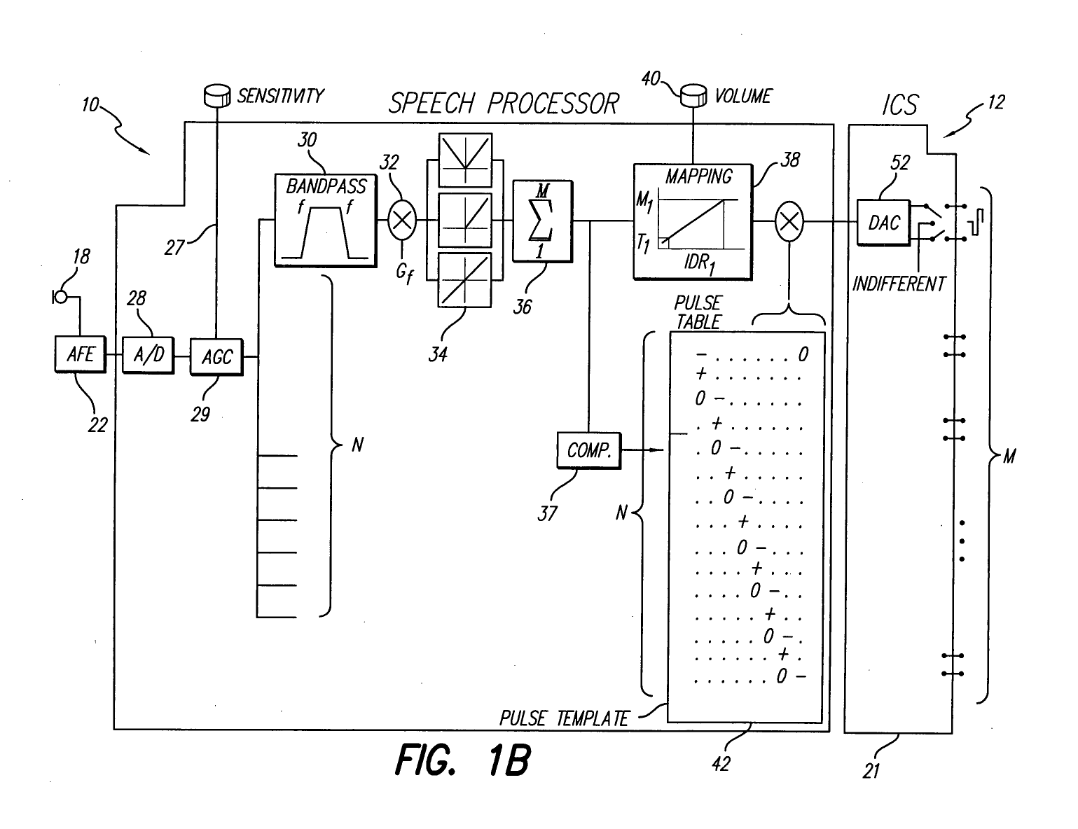

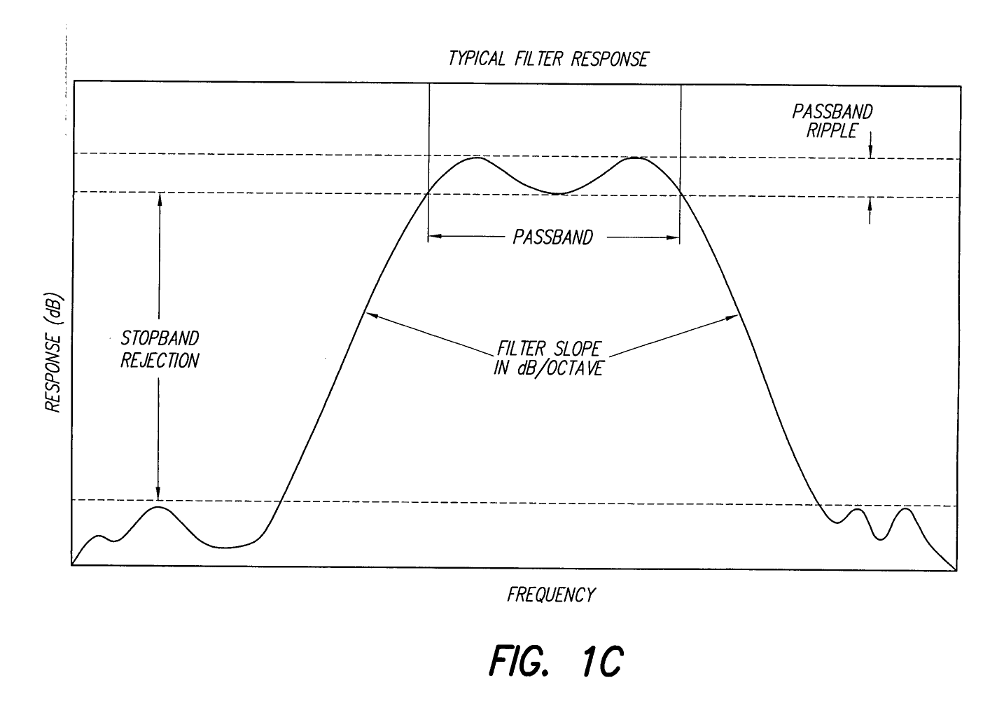

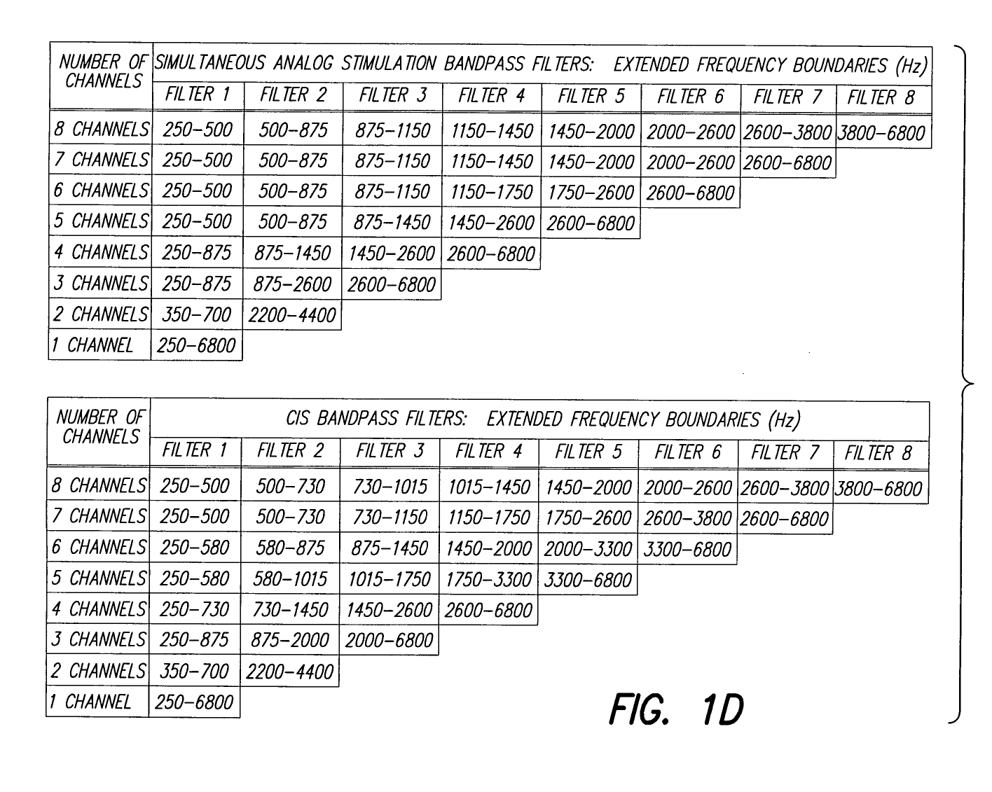

Turning to FIG. 1A, a cochlear stimulation system is shown that includes a speech processor portion 10 and a cochlear stimulation portion 12. The speech processor portion 10 includes a speech processor (SP) 16 and a microphone 18. The microphone 18 may be connected directly to the SP 16, or may be coupled to the SP 16 through an appropriate communication link 24. The cochlear stimulation portion 12 includes an implantable cochlear stimulator (ICS) 21, and an electrode array 48. The electrode array 48 is adapted to be inserted within the cochlea of a patient. The array 48 includes a multiplicity of electrodes, e.g., sixteen electrodes, spaced along its length that are selectively connected to the ICS 21. The electrode array 48 may be substantially as shown and described in U.S. Pat. No. 4,819,647, incorporated herein by reference, or U.S. Pat. No. 6,129,753, also incorporated herein by reference. Electronic circuitry within the ICS 21 allows a specified stimulation current to be applied to selected pairs or groups of the individual electrodes included within the electrode array 48 in accordance with a specified stimulation pattern, defined by the SP 16.

The ICS 21 and the SP 14 are linked together electronically through a suitable communications link 14 that allows power and control signals to be sent from the SP16 to the ICS 21, and that (in some embodiments) allows data and status signals to be sent from the ICS 21 to the SP 16. The details of such communication link 14 are not important for purposes of the present invention. In some embodiments, i.e., where the ICS 21 and electrode array 48 are implanted within the patient, and the SP16 and microphone 18 are carried externally (non-implanted) by the patient, the link 14 may be realized by an antenna coil in the ICS and an external antenna coil coupled to the SP. In such embodiment, i.e., when the ICS has been implanted, the external antenna is positioned so as to be aligned over the location where the ICS is implanted, allowing such coils to be inductively coupled to each other, thereby allowing information (e.g., the magnitude and polarity of a stimulation current) and power to be transmitted from the speech processor 16 to the ICS 21. In other embodiments, i.e., where both the SP 16 and the ICS 21 are implanted within the patient, the link 14 may be a direct wired connection, or other suitable link, e.g., as shown in U.S. Pat. No. 6,308,101, incorporated herein by reference.

The microphone 18 senses acoustic signals and converts such sensed signals to corresponding electrical signals. The electrical signals are sent to the SP 16 over a suitable electrical or other link 24. The SP 16 processes these converted acoustic signals in accordance with a selected speech processing strategy in order to generate appropriate control signals for controlling the ICS 21. Such control signals specify or define the polarity, magnitude, location (which electrode pair receives the stimulation current), and timing (when the stimulation current is applied to the electrode pair) of the stimulation current that is generated by the ICS.

It is common in the cochlear stimulator art to condition the magnitude and polarity of the stimulation current applied to the implanted electrodes of the electrode array 48 in accordance with a specified speech stimulation strategy. Such speech stimulation strategy involves defining a pattern of stimulation waveforms that are to be applied to the electrodes as controlled electrical currents. Before describing the Frequency Modulated Stimulation (FMS) strategy of the present invention, it will first be helpful to review the stimulation strategies commonly used in prior cochlear implant systems.

If multiple electrode pairs exist, as is the case with a multichannel cochlear stimulators, then the types of stimulation patterns applied to the multiple channels may be conveniently categorized as: (1) simultaneous stimulation patterns, or (2) non-simultaneous stimulation patterns. Simultaneous stimulation patterns may be “fully” simultaneous or partially simultaneous. A fully simultaneous stimulation pattern is one wherein stimulation currents, either analog or pulsatile, are applied to the electrodes of all of the available channels at the same time. A partially simultaneous stimulation pattern is one wherein stimulation currents, either analog or pulsatile, are applied to the electrodes of two or more channels, but not necessarily all of the channels, at the same time. Examples of each type are given below.

Analog waveforms used in analog stimulation patterns are typically reconstructed by the generation of continuous short monophasic pulses (samples). The sampling rate is selected to be fast enough to allow for proper reconstruction of the temporal details of the signal. An example of such a sampled analog stimulation pattern is a simultaneous analog sampler (SAS) strategy.

Current pulses applied in pulsatile stimulation patterns are generally biphasic pulses applied to the electrodes of each channel. The biphasic pulse has a magnitude (e.g., amplitude and/or duration) that varies as a function of the sensed acoustic signal. (A “biphasic” pulse is generally considered as two pulses: a first pulse of one polarity having a specified magnitude, followed immediately, or after a very short delay, by a second pulse of the opposite polarity having the same total charge, which charge is the product of stimulus current times duration of each pulse or phase.) For multichannel cochlear stimulators, it is common to sample the acoustic signal at a rapid rate, and apply a biphasic stimulation pulse in sequence (i.e., non-simultaneously) to each of the pairs of electrodes of each channel in accordance with a specified pattern and cycle time, with the magnitude of the stimulation current being a function of information contained within the sensed acoustic signal at a given (e.g., the most recent) sample time. An example of such sequential, non-simultaneous stimulation pattern is a continuous interleaved sampler (CIS) strategy.

It is important to recognize that in between the two extremes of fully simultaneous stimulation patterns (wherein analog stimulation currents are continuously applied to all channels, e.g., using the SAS strategy) and non-simultaneous pulsatile patterns (wherein biphasic pules are applied in a specified sequence to all channels without time overlap, e.g., using the CIS strategy), there are a great number of other stimulation patterns that may be formulated. Such other simulation patterns may prove more efficacious for a given patient than either of the SAS or CIS strategies.

The FMS strategy of the present invention, as explained more fully below, is preferably implemented as a non-simultaneous pulsatile pattern in order to conserve power, but the pulsatile pattern employed is vastly different than the pulsatile pattern employed in a CIS strategy. The FMS strategy generates non-simultaneous pulses based on the presence of signals within each frequency band and applies these pulses to an appropriate region of the cochlea corresponding to such frequency band, but correlates and synchronizes the delivery of such pulses with the refractory period of the cochlea, e.g., as synchronized (more or less) with the vibrations of the basilar membrane.

An important feature of the FMS strategy of the present invention is that non-simultaneous biphasic pulsatile stimulation pulses are used, as in CIS, to reduce channel interaction and preserve power. In order to avoid the simultaneous application of the pulses, a first-in-first-out (FIFO) buffer, or equivalent, may be used so that the first channel which requires a pulse, receives the pulse, while the other channels wait their turn to receive a pulse.

Turning next to FIG. 1B, a partial functional block diagram of the SP 16 and the ICS 21 of the system of FIG. 1A is shown. It is to be emphasized that what is shown in FIG. 1B depicts the functions that are carried out by either the SP 16 or the ICS 21. The actual electronic circuitry that is used to carry out these functions is not critical to the present invention, although a representation of circuitry that may be used for this function is shown in the previously cited patents and patent applications. It should also be pointed out that the particular functions shown in FIG. 1B are representative of a preferred type of signal processing strategy for use with the present invention (which divides the incoming signal into frequency bands, and independently processes each band).

In FIG. 1B, it is seen that the speech processing portion 10 includes a microphone 18 that senses acoustical information and converts it to electrical signals. These signals are then amplified in audio front-end (AFE) circuitry 22. The amplified audio signal is then converted to a digital signal by analog-to-digital (A/D) converter 28. The resulting digital signal is then subjected to automatic gain control (AGC) processing using a suitable AGC algorithm 29. The function of the AGC algorithm is to compress the dynamic range of the speech signals so as to provide a more consistent overall level of stimulus to the electrodes, as well as to equalize the level between close and more distant speakers in a given area, e.g., within a room. The AGC algorithm 29 operates by measuring the volume level of the signal and using the measurement result to control a variable gain stage. The gain is controlled by two loops, where the lowest control voltage of the two loops is selected. One loop performs syllabic compression by responding slowly to sounds above about 55 dB SPL, the second loop performs as a compression amplifier by responding quickly to sounds over about 67 dB SPL. [For a discussion of sound levels, and a definition of dB SPL, see “Moore, Brian C. J., “An Introduction to the Psychology of Hearing”, Fourth Edition, pp. 9-12 (Academic Press 1997).] A sensitivity control 27 is coupled to the AGC circuit 29. The sensitivity control 27 may be either a dial or remote control, preferably a remote control, and may vary either front-end gain and/or AGC parameters.

As seen in FIG. 1B, after processing by the AGC algorithm 29, the signal is processed in one of a multiplicity of digital signal processing channels. For example, eight separate analysis channels may be used, each responding to a different frequency content of the sensed acoustical signal. In other words, the incoming signal is divided into a multiplicity of N frequency channels, as defined by a bank of respective bandpass or other filters 30. Note that the lowest frequency filter may be a lowpass filter, and the highest frequency filter may be a high-pass filter.

Typical characteristics of the passband filters are illustrated as shown in FIG. 1C. All of the filters preferably have a maximum pass band ripple of 3 dB, a stop band rejection of at least 50 dB, a filter slope of 39 dB/octave, and internal filter noise of at least 50 dB below the signal.

The gain of each filter is equal for all channels by default, but may be modified by a user in ±10 dB increments, if desired. In FIG. 1B, the gain associated with the filter stage is functionally illustrated as a multiplication element 32, driven by the gain of the filter G_{f}. In an actual hardware implementation, as is known to those of skill in the art, the filtering and gain functions are typically carried out in a common amplifier/filter stage. In an actual software implementation, as is also known in the art, the filtering and gain functions may be performed sequentially, as shown in FIG. 1B.

One type of frequency map for the filters is shown in FIG. 1D. As seen in FIG. 1D, a slightly different frequency map is used by default for SAS than is used for CIS. The FMS strategy of the present invention may use either frequency map shown in FIG. 1D, or other frequency maps as are determined best suited for the needs of a particular patient or group of patients. That is, it is to be understood that other frequency maps may be readily used and new ones defined, as required, for the FMS speech processing strategy of the present invention.

At this point, it should be noted that the functional scheme illustrated in FIG. 1B offers a great deal of flexibility in order to allow different signal processing strategies to be employed. The full capability of the circuitry illustrated functionally in FIG. 1B is more fully described in U.S. Pat. No. 6,289,247, previously incorporated herein by reference, and will not be repeated herein. One of the advantages associated with the FMS strategy of the present invention is that much of the signal processing illustrated in FIG. 1B need not be performed. Hence, the signal processing required is simpler to perform and may be carried out using less power.

The FMS strategy employed by the invention, in its simplest form, may be implemented as shown in FIG. 2. As seen in FIG. 2, the FMS strategy simply divides the incoming sound signal, obtained from microphone 18 through front end (FE) circuitry 25, into separate frequency bands, e.g, using the bandpass filters 30a through 30n. Once such frequency separation has been performed, the only signal processing that need take place is some relatively simple FM processing, carried out by FM Processing circuitry 35a through 35n. (Here, it is to be understood that the term “circuitry” may include hardware components, firmware components, software programs, or combinations thereof.)

The output or result obtained from the FM Processing circuitry 35a-35n for each frequency band or channel is a trigger pulse, T, that is generated whenever a biphasic stimulation pulse is to be applied to an appropriate electrode, 54a-54n, of the electrode array 48 that has been inserted into the cochlea of the patient. Such trigger pulse is sent to a corresponding pulse generator, 52a-52n, which pulse generator generates a biphasic stimulation pulse, P, that is directed to the corresponding electrode 54a-54n of the electrode array. The frequency band or channel wherein the lowest frequencies are processed, comprising BPF₀ 30a, FM Processing Circuity 35a and pulse generator circuitry 52a, applies its stimulation pulses to the most distal electrode 54a. Electrode 54a comprises the one that is inserted deepest into the cochlea, where the nerves are located that respond to the lowest frequency signals. Likewise, the frequency band or channel wherein the highest frequencies are processed, comprising BPF_{N} 30n, FM Processing Circuity 35n and pulse generator circuitry 52n, applies its stimulation pulses to the most proximal electrode 54n. Electrode 54n comprises the one that is inserted shallowest into the cochlea, where the nerves are located that respond to the highest frequency signals. A plurality of additional electrodes, not shown in FIG. 2, reside in the cochlea intermediate the most distal electrode 54a and the most proximal electrode 54n where stimulation pulses from other frequency bands or channels may be sent for stimulating other nerves that are located to respond to frequency signals that are intermediate the highest and lowest frequency signals.

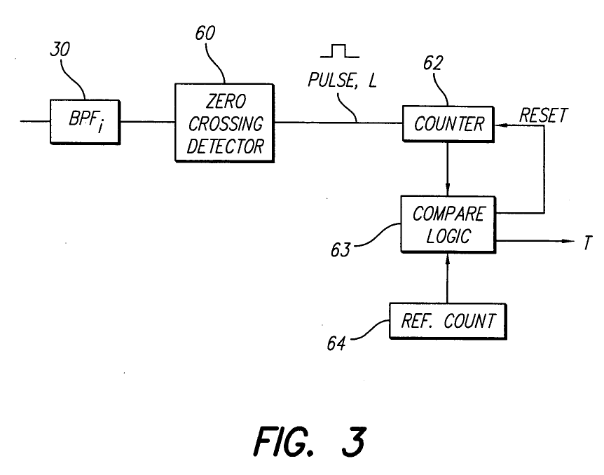

FIG. 3 shows one way that the FM processing circuits 35a-35n of FIG. 2 may be implemented in order to generate the trigger pulse T. As seen in FIG. 3, the output signal from the bandpass filter BPFᵢ of a frequency band or channel is directed to a zero crossing detector circuit 60. Such zero crossing detector circuit 60 is configured to output a pulse L for each cycle of the signal received from the bandpass filter, BPFᵢ. As such, the detector 60 may generate a pulse L for each positive-to-negative zero crossing of the incoming signal. Alternatively, the detector 60 may generate a pulse L for each negative-to-positive zero crossing of the incoming signal. Or, as explained below, the detector 60 may detect all zero crossings, and appropriate adjustments may be made in the circuitry that follows to account for the fact the pulse L occurs every ½ cycle, rather than every cycle.

Still alternatively, the zero crossing detector 60 may comprise a peak detector, in which case the detector 60 may generate a pulse L for each positive peak, or each negative peak, of the incoming signal.

The pulses L are counted in a counter 62. The contents are the counter 62 and compared, using compare logic circuitry 63, to a reference count held in a reference count register 64. The reference count varies depending upon the frequency band or channel, and is selected so that the trigger pulse T is generated when the number of counted pulses L represents a time interval that is more or less equal to the refractory period of the cochlea. When the trigger pulse T is generated, the counter 62 is reset, and the process repeats. In this manner, each region of the cochlea (where each region is known to have nerves associated therewith that, when triggered, send a nerve impulse to the brain through the auditory nerve that represents a sound within a certain frequency range, and wherein one or more electrodes are positioned within such region) receives a biphasic stimulus pulse when the corresponding frequency band of the incoming signal indicates continuous activity (i.e., spectral components) within that frequency band at a rate that is approximately no greater than, or less than, the refractory period of the cochlea.

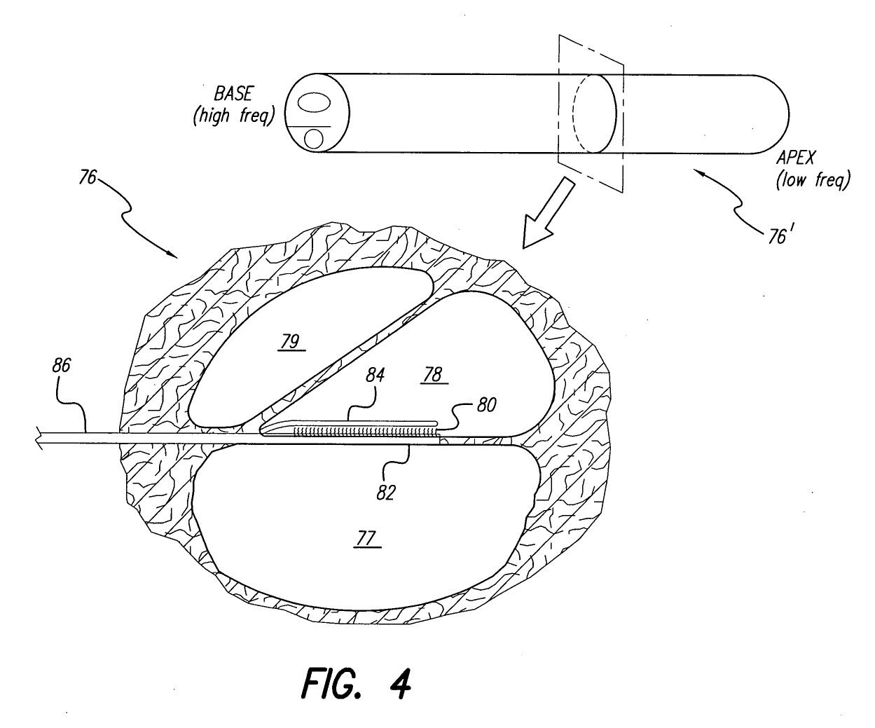

Turning next to FIG. 4, there is shown a schematic representation of a cross sectional view of a human cochlea 76. The cochlea 76 includes three separate channels that traverse the length of the cochlea. These channels are the scala tympany 77, the scala media 78, and the scala vestibuli 79. As is known, the natural neural firings of a healthy cochlea are caused by movement of the hair cells 80, which are located along the basilar membrane 82. The movement of the hair cells is caused by vibrations set up in the fluid that fills the cochlear channels by sound pressure waves that make their way through the outer and middle ears.

The healthy cochlea delivers sound, by first splitting the sound into frequency components, using the basilar membrane 82, in a tonatopic organization. The base end of the basilar membrane 82 filters out the high frequencies (i.e., senses and responds to the high frequencies), and the apical end filters out the low frequencies (i.e., senses and responds to the low frequencies). The basilar membrane 82 promotes the activation of neurons by bending the hair cells 80 against the tectorial membrane 84, which is adjacent the basilar membrane 82, during the upward motion of each vibration cycle. When a neuron is activated by the bending of a hair cell(s) 80, a nerve signal is generated and passed to the brain via the acoustic nerve 86. The acoustic nerve 86 comprises a bundle of individual nerve fibers. In the low frequencies, the neuron firings are phase aligned to each peak of the vibration. In the high frequency region of the cochlea, the neural firings are aligned with integer multiples of the basilar membrane vibration periods.

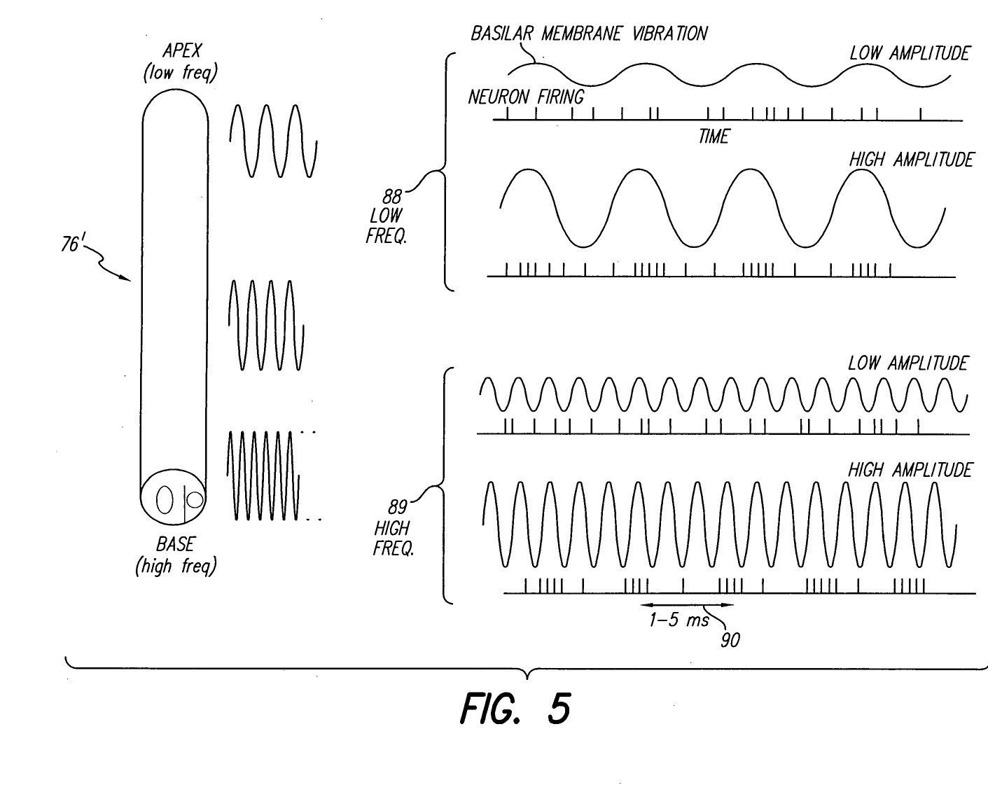

The normal neural firing patterns that occur within a healthy cochlea are illustrated in FIG. 5. On the left side of FIG. 5 is shown a schematic representation of the human cochlea 76′ (which cochlea is normally in a spiral shape) as it would appear if it were unwound and laid out in a straight path or line. (Such representation of a straightened out cochlea 76′ is also present at the top right hand side of FIG. 4.)

The cochlea 76′ responds (senses) to low frequencies at its apex, and responds (senses) high frequencies near its base. A diagrammatic representation of low and high amplitude low frequency signals are presented within the bracket 88 of FIG. 5. Similarly, a diagrammatic representation of low and high amplitude high frequency signals are presented within the bracket 89 of FIG. 5. As seen in the diagrams contained within either of these brackets 88 or 89, a low amplitude signal results in less neurons firing than does a high amplitude signal.

FIG. 5 further shows a time period, or window, 90 located across the bottom of the figure that is labeled as being between about 1 to 5 milliseconds (ms) in duration. This time period or window 90 represents the refractory period of the majority of the neurons associated with the hair cells of the cochlea. That is, once a neuron fires as a result of a particular hair cell bending in response to sound vibrations, it takes a certain period of time before the neuron can recover and be ready to fire again. This recovery period of the neuron is referred to herein as the “refractory period” (or “RP”) of the cochlea. Typically, as shown in FIG. 5, the refractory period 90 is on the order of 1-5 ms. Moreover, as is also shown in FIG. 5, it is seen that the neural responses tend to phase align to the signal along the basilar membrane (low frequencies).

This phenomenon—of having neural responses align to the signal along the basilar membrane—is predictable from physiology. The problem is that no neuron can physically fire faster than with a period of about 1 ms. Moreover, at any given location along the basilar membrane, the average period or duration between neural firings is about 1-5 ms, which is the refractory period (RP) of the cochlea. The present invention phase aligns the neural firings at any location along the length of the cochlea with those of the basilar membrane by determining how many periods N of an incoming signal within a given frequency band fit within the RP, and then triggering a stimulation pulse (to in turn trigger a neural firing) in that region of the cochlea only after N periods of the incoming signal.

Thus, the number of periods N that are counted off (using, e.g., a zero crossing detector 60 as shown in FIG. 3) for each frequency band is
N=RP*Fc
where RP is the refractory period of the cochlea and Fc is the center frequency of the frequency band of interest. Another way of looking at this is to determine how many periods of Fc will fit into the RP, so that
N=RP/(1/Fc)
where 1/Fc is the period of oscillation of Fc.

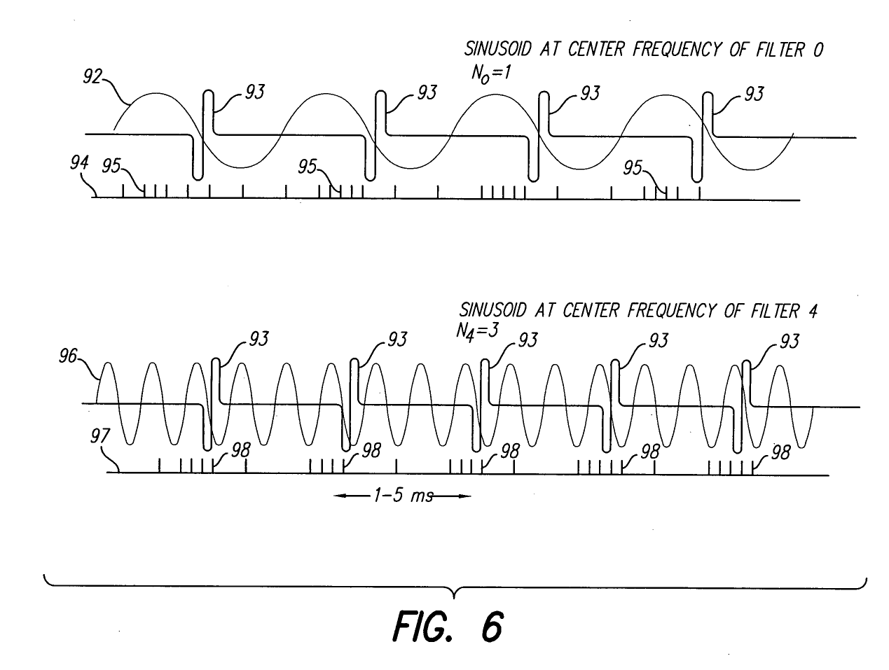

FIG. 6 illustrates one implementation of the Frequency Modulated Stimulation (FMS) strategy of the present invention. As seen in FIG. 6, the top waveform 92 represents a sound signal at a low frequency of a moderate-to-high amplitude. The neural firings associated with such low frequency signal are represented by the small vertical lines 95 spread across the horizontal time line 94. At each positive-to-negative zero crossing of the low frequency sound signal 92, a biphasic pulse 93 is generated. This is because, for this frequency band, the filter for which is labeled Filter 0 (a low frequency), the number of periods N of the incoming signal which fit into the RP (assumed to be 0.003 sec., or 3 ms) is 1, or N₀=1.

In contrast, the bottom waveform 96 in FIG. 6 represents a sound signal at a higher frequency, also having a moderate-to-high amplitude. the neural firings associated with such higher frequency signal are represented by the small vertical lines 98 spread across the horizontal time line 97. At every 4^{th} positive-to-negative zero crossing of the higher frequency sound signal 96, a biphasic pulse 93 is generated. This is because, for this frequency band, the filter for which is labeled Filter 4, the number of periods N of the incoming signal which fit into the RP (assumed to be 0.003 sec., or 3 ms) is 3, or N₄=3.

Thus, it is seen that the technique or algorithm used to generate stimulation pulses in accordance with the implementation of the invention depicted in FIG. 6 is simple and straight forward and may be stated as follows:
(1) if, for a given frequency band, having Filterᵢ, the Nith positive-to-negative zero crossing has been reached, the output, OUT, is a biphasic pulse having an amplitude that is equal to the maximum filter output amplitude since the last output, where
   Ni=0.03 sec*CenterFrequency(Filterᵢ); and
(2) otherwise, OUT=zero.

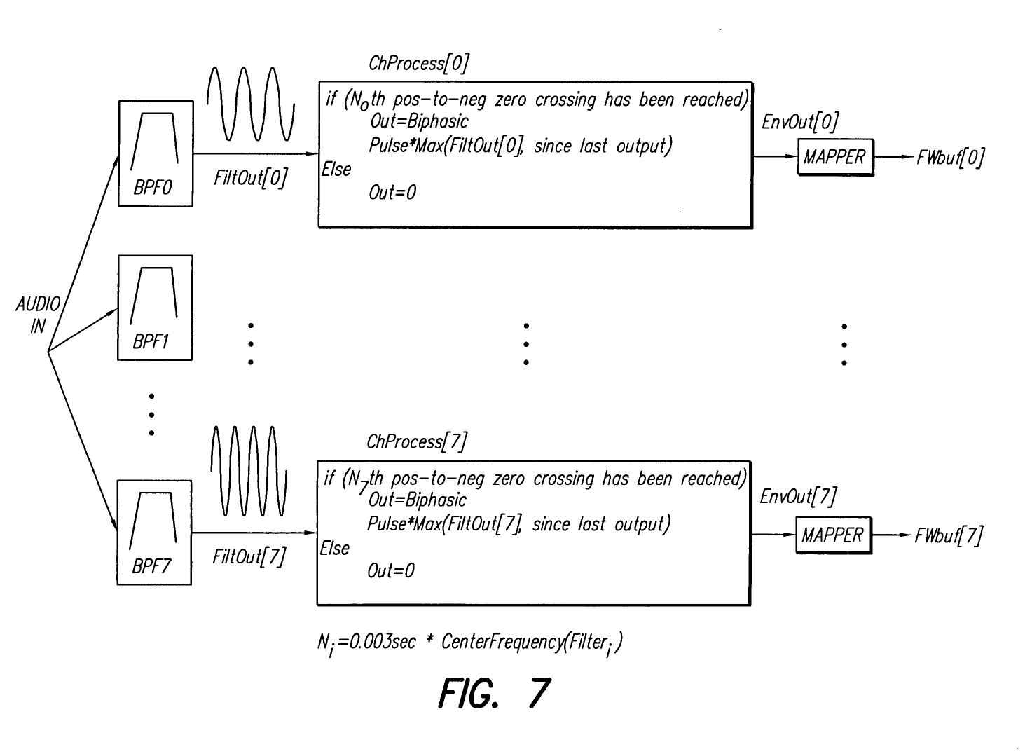

Turning next to FIG. 7, there is shown a functional diagram that depicts how the FMS strategy of the invention emulates the normal neuron firing pattern of the cochlea. As seen in FIG. 7, eight frequency channels are used, and the incoming audio signal is thus divided into eight frequency bands by eight filters, BPF0, BPF1, . . . BPF7. The respective output of each filter, FiltOut[0], FilterOut[1], . . . FilterOut[7] is presented to corresponding processing circuitry for that channel, ChProcess[0], ChProcess[1], . . . ChProcess[7]. Each channel processing circuitry carries out substantially the same processing algorithm in order to generate an output signal, EnvOut[i], for that channel (the ith channel). The value of this output signal may be expressed as:

if (Nith pos-to-neg zero crossing has been reached), then
EnvOut[i]=BiphasicPulse*Max(FiltOut[i], since last output
Else,
EnvOut[i]=0.
where
Ni=RP*CenterFrequency(BPFi)

The value of the refractory period, RP, may be determined empirically, or from the literature, or assigned a value suitable for a particular patient. For the example shown in FIG. 7, the RP is assumed to be 0.003 seconds (3 ms).

By way of example, assume that channel six filter, BPF5, has a center frequency Fc of 1170 Hz. Also, assume that the RP is 3 ms. Then, the number of periods that should occur before firing a stimulation pulse is:
N=0.003*1170=3.51.
But, since N must be an integer, the computed value of N is rounded off to arrive at a “rounded value” of N, which also may be referred to as “the rounded number of cycles” of
N≈4.
Thus, for channel six, a biphasic stimulation pulse would be generated for every 4 periods of the incoming signal. On average, this would be approximately every 3 ms.

Still referring to FIG. 7, it is seen that the EnvOut[i] signal is applied to a corresponding Mapper circuit, Map[i], which directs it to an appropriate electrode, or combinations of electrodes, for presentation to the cochlea.

The amplitude of the biphasic stimulation pulse may be determined in various ways. The algorithm described above uses the maximum amplitude obtained from the output of the BPFith filter since the last stimulation pulse was generated. This approach thus uses a rough approximation of the amplitude of the envelope of the incoming audio signal for that channel. Other approaches may also be used, such as a running average of the BPFith output, the most recent output of the BPFith filter, or the like. Any technique that provides a consistent amplitude for the biphasic stimulation pulse that is sufficiently strong to fire or trigger a neuron when presented is adequate for purposes of practicing the invention. This amplitude may be derived from or related to the amplitude, or energy content, or other relevant parameter, of the incoming audio signal for that particular channel, or may be derived from testing or fitting data. (Here, “fitting” refers to the process used to adjust or tune the parameters of an implantable cochlear stimulator once it has been implanted.)

It should also be noted that the refractory period, RP, may not be constant, nor static. Rather, the RP (at least for most patients) appears to be modulated between about 1 and 5 ms according to the amplitude of the incoming signal. Thus, in a healthy cochlea, a loud signal will, on average, produce a neuronal response every 1 ms, but a quiet signal of the same frequency will produce a neuronal response only about every 5 ms, but sill always at an integer multiple of the signal period.

Hence, in accordance with one preferred embodiment of the present invention, the RP value is treated as a variable and is modulated in accordance with the amplitude of the filtered signal. This causes a stimulation pulse to be generated sooner for a loud signal (i.e., fewer integer multiples of the signal period) than for a quiet signal of the same frequency.

### EXAMPLE 1

A first patient, who had previously been using a CIS strategy, was fitted with a cochlear implant of a first type using the FMS strategy of the present invention. It is noted that during the fitting process of most cochlear implants, regardless of the strategy that is employed, “M” and “T” levels are measured in order to adjust the intensity of the electrical stimuli that are applied to the patient. See, e.g., U.S. Pat. No. 6,289,247, previously incorporated herein by reference, for a more detailed explanation of the overall fitting process, including a more detailed description of “T” and “M” levels. A “T” level is a stimulation threshold level below which the patient is generally not able to perceive the stimulation, i.e., neural firing does not occur. An “M” level is a stimulation intensity that is generally comfortable for the patient, i.e., neural firing does occur, but not at a level that is too intense or uncomfortable. The “M” and “T” levels, especially the “M” levels, once set, can thereafter be used as a quasi figure of merit of how effectively the stimulator is able to trigger a neural response on a particular channel. That is, a relatively low “M” level is usually considered a good goal because it means effective stimulation is occurring on that channel at relatively lower power levels.

The RP for the first patient was first set to 3 ms. The “M” levels of the patient decreased from 200 to 130 on average. The patient reported that the sounds were “good”, with good speech perception. The patient really liked listening to Vivaldi using this FMS strategy at this setting. However, the patient reported that her own voice sounded “scratchy”, and observers thought her voice sounded “scratchy” as well. The power expended was 3.8 times better than when using the CIS strategy.

The RP of this first patient was then set to 6 ms. The “M” levels were on average the same as had been used on the CIS strategy. Some “rattle” and “thunder” sounds were present, but were eliminated by cutting out channels 1 and 2. Speech perception was acceptable, but the patient did not like it as well once channels 1 and 2 were eliminated. The power expended was 6.5 times better than when using the CIS strategy, after elimination of channels 1 and 2.

### EXAMPLE 2

A second patient, also previously using a CIS strategy, and having a second type of cochlear implant, was fitted using the FMS strategy of the present invention, with the RP set to 3 ms. The “M” levels decreased from 676 to 480 on average. The perceived sounds were good, with good speech perception. Music was reported as sounding more “full”. A voice of a nearby person was reported as sounding somewhat “scratchy”. The power expended was 2.8 times better than was achieved using the CIS strategy.

The RP of this second patient was then modulated to be 6 ms for low intensity sounds and 3 ms for high intensity sounds. The “M” levels decreased from 676 to 542 on average. The patient reported that the implant worked well, and said that music “sounds great”. The patient wanted to continue using the FMS strategy on an extended basis. No “scratchiness” or other complaints were noted. The power expended was 3.0 times better than when using the CIS strategy.

As described above, it is thus seen that the present invention provides frequency modulated stimulation strategy for use in a multichannel cochlear implant that provides a more natural sound, like an SAS strategy, but at a lower power consumption, like a CIS strategy.

It is further seen that the FMS strategy provided by the invention offers improved performance and perception due to the manner in which the electrical stimuli are presented to the tonotopically arranged cochlea. Lower “M” levels are achieved because the neurons are correlated to each firing, since the average presentation rate is lower than the refractory period of the neurons.

It is also seen that the FMS strategy provided by the invention allows a significant power savings to be achieved due to the lower rate at which the electrical stimuli are presented to the cochlea.

While the invention herein disclosed has been described by means of specific embodiments and applications thereof, numerous modifications and variations could be made thereto by those skilled in the art without departing from the scope of the invention set forth in the claims.

What is claimed is:

1. A method of stimulating the cochlea using a frequency modulated stimulation strategy comprising:
(a) dividing incoming sound signals into at least four frequency bands, each frequency band covering a specified portion of the audio spectrum, each frequency band providing an output signal that varies as a function of the sound components present within that frequency band;
(b) for each frequency band, monitoring the number of cycles of the output signal of that frequency band;
(c) for every Nth cycle of the output signal of each frequency band, stimulating a respective region of the cochlea, where N represents an integer that defines the rounded number of cycles of the output signal for that frequency band that occur during a predetermined refractory period of the cochlea, whereby each region of the cochlea is stimulated no more than once during the predetermined refractory period as a function of the sound components that occur within the frequency band associated with that region of the cochlea.

2. The method set forth in claim 1 wherein step (a) includes dividing the incoming sound signals into at least eight frequency bands.

3. The method set forth in claim 2 wherein the eight frequency bands include a first frequency band covering approximately 200-500 Hz, a second band covering approximately 500-875 Hz, a third band covering approximately 875-1150 Hz, a fourth band covering approximately 1150-1450 Hz, a fifth band covering approximately 1450-2000 Hz, a sixth band covering approximately 2000-2600 Hz, a seventh band covering approximately 2600-3800 Hz and an eighth band covering frequencies above 3800 Hz.

4. The method set forth in claim 1 wherein step (b) comprises counting the number of positive-to-negative zero crossings that occur in the output signal.

5. The method set forth in claim 1 wherein step (b) comprises counting the number of negative-to-positive zero crossings that occur in the output signal.

6. The method set forth in claim 1 wherein step (b) comprises counting the number of zero crossings that occur in the output signal and dividing by two.

7. The method set forth in claim 1 wherein the refractory period is modulated, having a shorter duration for higher intensity signals and a longer duration for lower intensity signals.

8. The method set forth in claim 1 wherein the modulated refractory period varies from about 1 ms to about 5 ms.

9. A method of stimulating a cochlea comprising:
(a) making an electrode array that is insertable into the cochlea, said electrode array having a flexible carrier on which n spaced-apart electrode contacts are located, wherein n is an integer of at least four, and wherein one of the electrode contacts comprises a distal electrode contact that is located at or near a distal end of the flexible carrier, and wherein another one of the electrode contacts comprises a proximal electrode contact that is located closest to a proximal end of the flexible carrier, and wherein all of the other electrode contacts are intermediate the distal electrode contact and the proximal electrode contact;
(b) inserting the electrode array into the cochlea so that the distal electrode contact is deepest into the cochlea, and the proximal electrode contact is shallowest into the cochlea;
(c) sensing sound external to the cochlea;
(d) frequency dividing the sensed sound into at least n frequency bands, wherein each frequency band provides an oscillating electrical output signal representative of the sensed sound having frequency components residing within that frequency band;
(e) counting the number of periods of the electrical output signal associated with each frequency band that occur within a prescribed time duration; and
(f) generating an electrical stimulus for each channel after the occurrence of a prescribed number of periods of the electrical output signal for that channel, and mapping the generated electrical stimulus to an appropriate one of the n spaced-apart electrode contacts corresponding to that channel, wherein the channel corresponding to the highest frequency band is mapped to the proximal electrode contact, the channel corresponding to the lowest frequency band is mapped to the distal electrode contact, and the channels corresponding to intermediate frequency bands are mapped to respective intermediate electrode contacts.

10. The method of claim 9 wherein the prescribed time duration during which the number of periods of the electrical output signal is counted comprises a refractory period of the cochlea.

11. The method of claim 10 further including modulating the refractory period to be shorter when the intensity of the electrical output signal associated with a given frequency band is high, and to be longer when the intensity of the electrical output signal associated with the given frequency band is low.

12. A cochlear stimulator for stimulating the cochlea in accordance with a frequency modulated stimulation strategy, comprising:
means for dividing incoming sound signals into a multiplicity of frequency bands, each frequency band covering a respective portion of the audio spectrum, each frequency band providing an output signal that varies as a function of the sound components present within that frequency band;
means for monitoring the number of cycles of the output signal of each frequency band;
means for stimulating a respective region of the cochlea for every Nth cycle of the output signal of each frequency band, where N represents an integer that roughly defines the number of cycles of the output signal for that frequency band that occur during a predetermined refractory period of the cochlea, whereby each region of the cochlea is stimulated no more than once during the predetermined refractory period as a function of the sound components that occur within the frequency band associated with that region of the cochlea.

13. The cochlear stimulator of claim 12 further including means for modulating the predetermined refractory period as a function of the intensity of the incoming sound signals of each frequency band.

14. The cochlear stimulator of claim 13 wherein the means for modulating the refractory period comprises means for modulating the refractory period between a maximum value of about 5 ms and a minimum value of about 1 ms.

15. The cochlear stimulator of claim 13 wherein the multiplicity of frequency channels into which the incoming sound signals are divided comprises at least eight.
